(19) United States
(12) Patent Application Publication
(10) Pub. No.: US 2022/0354922 A1
(43) Pub. Date: Nov. 10, 2022

(54) GINGER DERIVED EXTRACELLULAR VESICLES AND USE THEREOF

(71) Applicants: KYUNGPOOK NATIONAL UNIVERSITY INDUSTRY-ACADEMIC COOPERATION FOUNDATION, Daegu (KR); ANDONG NATIONAL UNIVERSITY INDUSTRY-ACADEMIC COOPERATION FOUNDATION, Andong-si, Gyeongsangbuk-do (KR)

(72) Inventors: Moon-Chang BAEK, Daegu (KR); Young-Eun CHO, Daejeon (KR); Dong Ha KIM, Andong-si, Gyeongsangbuk-do (KR)

(73) Assignees: KYUNGPOOK NATIONAL UNIVERSITY INDUSTRY-ACADEMIC COOPERATION FOUNDATION, Daegu (KR); ANDONG NATIONAL UNIVERSITY INDUSTRY-ACADEMIC COOPERATION FOUNDATION, Andong-si, Gyeongsangbuk-do (KR)

(21) Appl. No.: 17/764,985

(22) PCT Filed: Sep. 25, 2020
(86) PCT No.: PCT/KR2020/013132

§ 371 Date: May 10, 2022

(30) Foreign Application Priority Data
Oct. 1, 2019 (KR) 10-2019-0121528
Sep. 25, 2020 (KR) 10-2020-0124687

(51) Int. Cl. A61K 36/9068; A23L 33/17; A23L 33/105; A61K 38/44; A61K 38/48

(52) U.S. Cl. CPC A61K 36/9068; A23L 33/17; A23L 33/105; A61K 38/44; A61K 38/48

(57) ABSTRACT

The present invention relates to extracellular vesicles derived from ginger (Zingiber officinale) and a use thereof, and provides: extracellular vesicles which are derived from ginger and have anticancer activity, a pharmaceutical composition and a health functional food composition which is for preventing or treating cancer and contains the extracellular vesicles as an active ingredient. The ginger-derived extracellular vesicles can penetrate into cancer cells, have excellent anticancer activity, such as inhibiting the proliferation, infiltration and migration of cancer cells, and thus may be provided as an effective anticancer composition.

### CROSS REFERENCE TO PRIOR APPLICATIONS

This application is the 35 U.S.C. 371 national stage of international application PCT/KR2020/013132 filed on Sep. 25, 2020 which claims priority to Korean Patent Application Nos. 10-2019-0121528 filed on Oct. 1, 2019 and 10-2020-0124687 filed on Sep. 25, 2020. The entire contents of each of the above-identified applications are hereby incorporated by reference.

### TECHNICAL FIELD

The present disclosure relates to extracellular vesicles derived from ginger (Zingiber officinale) and a use thereof.

### BACKGROUND ART

Cancer is one of the incurable diseases that humanity needs to overcome, and huge capital is being invested in development for treating cancer worldwide. In Korea, it is the number one cause of death among Koreans since 1983, with more than 100,000 people diagnosed annually while more than 60,000 people are dying.

In particular, the incidence rate of breast cancer among Korean women has continued to increase since 1999, and in 2015, breast cancer took the first place in cancer incidence, overtaking thyroid cancer which had been the major cancer in women. Breast cancer has the ability to metastasize to form secondary cancer by easily migrating from the primary site to other sites via lymph nodes or blood vessels and is particularly known to selectively metastasize to bones or lungs, making the metastatic breast cancer patients difficult to be improved even with surgery and various therapies. Owing to recent medical developments, various methods such as surgical treatment, radiation treatment, and chemotherapy have been improved, ensuring 92.7% for the 5-year relative survival rate for breast cancer. Despite the high survival rate, the quality of life of patients may deteriorate due to the fear of recurrence and the physical and sociophysical symptoms that the patients go through during the treatment.

Due to difficult treatment methods at the time of onset and the deterioration in the quality of life after treatment, increased is people's interest in developing cancer prevention and treatment therapies that have fewer side effects while minimizing pain on the human body, unlike conventional chemotherapy which is to treat with anticancer drug, surgical treatment such as partial mastectomy (breast-conserving surgery) and total mastectomy, and radiotherapy which is to treat tumor using high-energy radiation. Accordingly, there have been a lot of interests in plant-derived natural substances that may be easily obtained from nature with fewer side effects and effectively treat cancer.

Extracellular vesicles (EVs) are substances formed during cellular activity and are also called nanovesicles with a size of 1/10⁹ m. The extracellular vesicles are divided into three groups, exosomes, microvesicles, and apoptotic bodies. The origin of exosomes is the endocytic pathway with the size of 30-100 nm and may be observed in body fluids such as blood and urine of multicellular eukaryotic organisms and in the culture medium of eukaryotic cells. The origin of microvesicles is plasma membrane, and the size thereof is 50-1,000 nm. The origin of apoptotic bodies is also plasma membrane, and the size thereof is 500-2,000 nm. Various clinical studies such as diagnosis, prognosis, and treatment of diseases are being conducted using the nanovesicles.

Recently, there is a report that nanovesicles are secreted from food, and thereamong, the anti-inflammatory effect of exosome-like nanoparticles in ginger has been reported, but the anticancer effect has not yet been reported.

### DISCLOSURE

### Technical Problem

An object of the present disclosure is to provide extracellular vesicles derived from a natural product.

Another object of the present disclosure is to provide an anticancer composition comprising the extracellular vesicles.

Another object of the present disclosure is to provide a method of producing the extracellular vesicles.

### Technical Solutions

In order to achieve the above object, example embodiments of the present disclosure provide an extracellular vesicle which is derived from ginger (Zingiber officinale) and has anticancer activity.

Example embodiments of the present disclosure provide a pharmaceutical composition for preventing or treating cancer comprising the extracellular vesicle as an active ingredient.

Example embodiments of the present disclosure provide a health functional food composition for preventing or ameliorating cancer comprising the extracellular vesicle as an active ingredient.

In addition, example embodiments of the present disclosure provide a method of producing ginger (Zingiber officinale)-derived extracellular vesicles, including preparing ground ginger, firstly centrifuging the prepared ground ginger at 250 to 750 g for 5 to 15 minutes, secondly centrifuging a supernatant obtained by the first centrifugation at 1,000 to 3,000 g for 10 to 30 minutes, thirdly centrifuging a supernatant obtained by the second centrifugation at 5,000 to 15,000 g for 15 to 45 minutes, and fourthly centrifuging a supernatant obtained by the third centrifugation at 50,000 to 150,000 g for 1 to 3 hours to obtain a pellet.

### Advantageous Effects

The ginger-derived extracellular vesicle according to example embodiments of the present disclosure may penetrate into cancer cells and have excellent anticancer activity, such as effective inhibition of proliferation, invasion, and migration of cancer cells. Accordingly, the ginger-derived extracellular vesicle may be provided as a pharmaceutical composition or a health functional food composition for preventing, ameliorating, or treating cancer diseases.

In addition, by using the ginger-derived extracellular vesicle according to example embodiments of the present disclosure rather than ingesting or using ginger as it is, it is possible to make up for shortcomings regarding pungent taste and flavor peculiar to ginger, long-term storage becomes easy, and excellent anticancer properties may be derived even in small amounts.

### BRIEF DESCRIPTIONS OF THE DRAWINGS

FIG. 1 schematically illustrates a method of isolating nanovesicles from ginger.

FIG. 2 is a graph of analyzing the size of ginger nanovesicles according to an experimental example of the present disclosure.

FIG. 3 is a graph showing a proliferation rate of breast cancer cells depending on the concentration of ginger nanovesicles according to an experimental example of the present disclosure.

FIG. 4 is a graph showing an effect of ginger nanovesicles on invasion of breast cancer cells depending on the concentration of ginger nanovesicles according to an experimental example of the present disclosure.

FIG. 5 shows a degree of migration of breast cancer cells depending on the concentration of ginger nanovesicles according to an experimental example of the present disclosure.

FIG. 6 is an image showing penetration of ginger nanovesicles into breast cancer cells according to an experimental example of the present disclosure.

FIG. 7 is a graph showing a proliferation rate of lung cancer cells depending on the concentration of ginger nanovesicles according to an experimental example of the present disclosure.

FIG. 8 shows a degree of migration of lung cancer cells depending on the concentration of ginger nanovesicles according to an experimental example of the present disclosure.

FIG. 9 is a graph identifying a DPPH radical scavenging ability of ginger nanovesicles according to an experimental example of the present disclosure.

### BEST MODE

Hereinafter, the present disclosure will be described in detail.

The present inventor has completed the present disclosure by determining that the nanovesicles isolated from ginger effectively inhibit proliferation, invasion, and migration of breast cancer cells or lung cancer cells.

An example embodiment of the present disclosure provides extracellular vesicles having anticancer activity.

The extracellular vesicles may be derived from ginger (Zingiber officinale).

The term “ginger (Zingiber officinale)” as used herein refers to a monocotyledonous perennial plant belonged to Zingiberaceae in the order Zingiberales and is also a medicinal plant having rhizomes in a yellow lumpy shape with a spicy taste and fragrant smell. In oriental medicine, dried rhizomes are used as medicines and are known to be effective in treating chills, fever, headache, vomiting, chronic cough, and phlegm caused by a cold, as well as abdominal pain, diarrhea, and abdominal distension due to food poisoning.

The ginger may be a rhizome, a leaf, a flower, or a mixture thereof, and preferably a rhizome portion may be used, but is not limited thereto.

The term “extracellular vesicle (EV)” as used herein refers to a small membranous vesicle secreted from various cells, represents a vesicle that is released out to the extracellular environment due to the fusion of polycystic bodies and the plasma membrane, and may be defined as a nanovesicle.

In the present disclosure, the extracellular vesicles may include exosomes and microvesicles, but are not limited thereto.

The extracellular vesicles may be nanovesicles having an average particle diameter of 50 to 300 nm, and preferably may have an average particle diameter of 100 to 200 nm, but is not limited thereto.

In the present disclosure, the extracellular vesicles may be isolated from ginger by a method such as ultracentrifugation, density gradient centrifugation, ultrafiltration, size exclusion chromatography, ion exchange chromatography, immunoaffinity capture, microfluidics-based isolation, exosome precipitation, total exosome isolation kit, or polymer based precipitation, but is not limited thereto.

Preferably, the extracellular vesicles may be obtained by centrifuging ginger, and more preferably, the extracellular vesicles are obtained by the first centrifugation of ground ginger at 250 to 750 g for 5 to 15 minutes, the second centrifugation of a supernatant obtained by the first centrifugation at 1,000 to 3,000 g for 10 to 30 minutes, the third centrifugation of a supernatant obtained by the second centrifugation at 5,000 to 15,000 g for 15 to 45 minutes, and the fourth centrifugation of a supernatant obtained by the third centrifugation at 50,000 to 150,000 g for 1 to 3 hours, and may be a pellet excluding a supernatant from the centrifugate obtained by the fourth centrifugation, but is not limited thereto.

The ground ginger may be ground by putting ginger in a phosphate buffer solution (PBS) but is not limited thereto. The ground ginger may also be directly prepared in the form of ginger juice or extracts, or obtained by purchasing commercially available ground ginger, ginger juice, or extracts thereof.

The extracellular vesicles obtained thereby may include one or more enzyme proteins selected from the group consisting of protease, cysteine proteinase, and glyceraldehyde-3-phosphate dehydrogenase, but is not limited thereto.

In the present disclosure, the extracellular vesicles may penetrate into cancer cells and have anticancer activity to inhibit proliferation, migration or invasion of cancer cells.

According to an experimental example of the present disclosure, it was confirmed that the extracellular vesicles penetrated into breast cancer cells or lung cancer cells, and it was also confirmed that proliferation, invasion and migration of the cancer cells were significantly inhibited.

Accordingly, the extracellular vesicles may be provided as a pharmaceutical composition or a health functional food composition for preventing or treating cancer.

An example embodiment of the present disclosure provides a pharmaceutical composition for preventing or treating cancer comprising the ginger-derived extracellular vesicles as an active ingredient.

The composition may include 0.001 to 50 parts by weight of the extracellular vesicles, but is not limited thereto.

The composition may prevent or treat cancer by penetrating into cancer cells to inhibit proliferation, migration or invasion of cancer cells.

The cancer may be a disease selected from the group consisting of breast cancer, lung cancer, liver cancer, stomach cancer, colorectal cancer, kidney cancer, bladder cancer, acute myeloid leukemia, acute lymphocytic leukemia, uterine cancer, ovarian cancer, laryngeal cancer, prostate cancer, thyroid cancer, head or neck cancer, brain cancer, and blood cancer, but is not limited thereto.

The pharmaceutical composition according to an example embodiment of present disclosure may be prepared according to a conventional method in the pharmaceutical field. The pharmaceutical composition may be combined with a pharmaceutically acceptable, appropriate carrier depending on the formulation, and if necessary, may be prepared by further including excipients, diluents, dispersants, emulsifiers, buffers, stabilizers, binders, disintegrants, and solvents. The appropriate carrier does not deteriorate the activities and properties of the ginger-derived extracellular vesicles according to an example embodiment of the present disclosure and may be selected differently depending on the administration type and formulation.

As the carrier, the excipient, and the diluent that may be included in the pharmaceutical composition, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil are used. When formulating the composition, diluents or excipients such as fillers, extenders, binders, wetting agents, disintegrants, and surfactants which are commonly used may be used for the formulation.

The pharmaceutical composition according to an example embodiment of the present disclosure may be applied in any formulation, and specifically, be used by being formulated into oral dosage formulation such as powder, granules, tablets, capsules, suspensions, emulsions, syrups, and aerosols, external preparations, suppositories, and sterile injection solutions according to conventional methods. Preferably, it may be used by being formulated into a unit dosage formulation suitable for oral administration.

More specifically, solid formulation among the oral dosage formulations is in the form of tablets, pills, powder, granules, and capsules to be prepared by mixing at least one or more excipients such as starch, calcium carbonate, sucrose, lactose, sorbitol, mannitol, cellulose, and gelatin, and lubricants such as magnesium stearate and talc may be included in addition to simple excipients. In addition, the capsule formulation may further include a liquid carrier such as fatty oil in addition to the above-mentioned substances.

A liquid formulation among the oral dosage formulations may be suspensions, solutions, emulsions, and syrups, and various excipients such as wetting agents, sweeteners, fragrances, and preservatives may be included in addition to water and liquid paraffin, which are commonly used simple diluents.

As the parenteral formulation, sterile aqueous solutions, non-aqueous solvents, suspensions, emulsions, injections, freeze-dried formulations, and suppositories may be included. As the non-aqueous solvents and suspensions, propylene glycol, polyethylene glycol, vegetable oils such as olive oil, and injectable esters such as ethyl oleate may be used. Witepsol, macrogol, Tween 61, cacao butter, laurin fat, and glycerogelatin may be used as a base of the suppositories. Any appropriate agent well known in the art may be used while it is not limited thereto.

In addition, the pharmaceutical composition according to an example embodiment of the present disclosure may further be added with an antioxidant to enhance therapeutic efficacy. As the antioxidant, compounds in the vitamin B group such as thiamin (vitamin B1), riboflavin (vitamin B2), niacin (vitamin B3), pantothenic acid (vitamin B5), pyridoxine (vitamin B6), and cobalamin (vitamin B12) as well as vitamin C, vitamin D, and vitamin E may be used, but are not limited thereto while all suitable formulations well known in the art may be used.

The pharmaceutical composition according to an example embodiment of the present disclosure may be administered in a pharmaceutically effective amount.

The term “pharmaceutically effective amount” as used herein refers to an amount sufficient to treat a disease at a reasonable benefit/risk ratio applicable to medical treatment while not causing side effects.

The effective dose level of the pharmaceutical composition may be differently determined depending on the purpose of use, the age, sex, weight and health status of a patient, the type of disease, the severity, the activity of a drug, the sensitivity to a drug, an administration method, administration duration, administration route and excretion rate, a treatment period, elements including drugs blended or used in combination with, and other factors well known in the medical field. For example, although not constant, generally 0.001 to 100 mg/kg, preferably 0.01 to 10 mg/kg, may be administered once to several times a day. The above dosage does not limit the scope of the present disclosure in any way.

The pharmaceutical composition according to an example embodiment of the present disclosure may be administered to any animal that cancer may be developed, and the animal may include, for example, not only humans and primates, but also livestock such as cattle, pigs, horses, and dogs.

The pharmaceutical composition may be administered in an appropriate administration route depending on the type of the formulation and may be administered via various routes, either oral or parenteral as long as it is able to reach a target tissue. The administration method is not particularly limited and may be conducted in a conventional methods such as oral, rectal or intravenous, muscle, skin application, respiratory inhalation, intrauterine dura mater or intracere-broventricular injection.

The pharmaceutical composition according to an example embodiment of the present disclosure may be used alone for the prevention or treatment of cancer or be used in combination with surgery or other drug treatment.

An example embodiment of the present disclosure provides a health functional food composition for preventing or ameliorating cancer including the ginger-derived extracellular vesicles as an active ingredient.

The composition may prevent or ameliorate cancer by penetrating into cancer cells to inhibit proliferation, migration, or invasion of cancer cells.

The cancer may be a disease selected from the group consisting of breast cancer, lung cancer, liver cancer, stomach cancer, colorectal cancer, kidney cancer, bladder cancer, acute myeloid leukemia, acute lymphocytic leukemia, uterine cancer, ovarian cancer, laryngeal cancer, prostate cancer, thyroid cancer, head or neck cancer, brain cancer, and blood cancer, but is not limited thereto.

Corresponding features may be substituted for the above-mentioned parts.

The term “health functional food” as used herein refers to food with high medical, clinical effects, which includes food manufactured and processed using raw materials or components having useful functionality for the human body according to Functional Foods for Health Act No. 6727, and is also processed to efficiently derive bioregulatory functions such as prevention of cancer, amelioration, body defense, immunity, and recovery for the purpose of the present disclosure in addition to nutrition supply.

The health functional food according to an example embodiment of the present disclosure may be prepared in the form of powder, granules, tablets, capsules, syrups or beverages for the purpose of preventing or ameliorating cancer. There is no limitation in the form that the health functional food may take, and the health functional food may be formulated in the same way as the pharmaceutical composition so as to be used as a functional food or added to various foods.

The health functional food may include any food in a conventional sense. For example, beverages and various drinks, fruits and processed foods thereof (canned fruit and jam), fish, meat and processed foods thereof (ham and bacon), breads and noodles, cookies and snacks, and dairy products (butter and cheese) are possible, and all functional foods in a conventional sense may be included. Food used as feed for animals may also be included.

The health functional food composition according to an example embodiment of the present disclosure may be prepared by further including food additives acceptable in food science and other appropriate auxiliary components commonly used in the art. The suitability as the food additive may be determined by the standards and criteria related to corresponding items according to the general rules and general test methods of Korean Food Additives Codex approved by the Ministry of Food and Drug Safety, unless otherwise stipulated. The items listed in the “Korean Food Additives Codex” may include, for example, chemical compounds such as ketones, glycine, calcium citrate, nicotinic acid, and cinnamic acid; natural additives such as persimmon pigments, licorice extracts, crystalline cellulose, kaoliang color, and guar gum; and mixed preparations such as sodium L-glutamate preparations, noodle-added alkali agents, preservative preparations, and tar color preparations.

The other auxiliary components may additionally include, for example, flavoring agents, natural carbohydrates, sweeteners, vitamins, electrolytes, coloring agents, pectic acid, alginic acid, organic acids, protective colloidal thickeners, pH adjusting agents, stabilizers, preservatives, glycerin, alcohols, and carbonating agents. In particular, monosaccharides such as glucose and fructose, disaccharides such as maltose and sucrose, polysaccharides such as dextrin and cyclodextrin, and sugar alcohols such as xylitol, sorbitol, and erythritol may be used as the natural carbohydrate, and natural sweeteners such as thaumatin and stevia extracts or synthetic sweeteners such as saccharin and aspartame may be used as the sweetener.

The effective dose of the extracellular vesicles contained in the health functional food according to an example embodiment of the present disclosure may be appropriately adjusted depending on the purpose of use thereof, such as prevention or amelioration of cancer.

The health functional food composition causes no side effects that may occur during long-term administration of general drugs by using food as a raw material and may be taken as an adjuvant for prevention or amelioration of cancer due to excellent portability.

In addition, an example embodiment of the present disclosure provides a method of producing ginger-derived extracellular vesicles.

In addition, the production method includes preparing ground ginger; firstly centrifuging the prepared ground ginger at 250 to 750 g for 5 to 15 minutes; secondly centrifuging a supernatant obtained by the first centrifugation at 1,000 to 3,000 g for 10 to 30 minutes; thirdly centrifuging a supernatant obtained by the second centrifugation at 5,000 to 15,000 g for 15 to 45 minutes; and fourthly centrifuging a supernatant obtained by the third centrifugation at 50,000 to 150,000 g for 1 to 3 hours to obtain a pellet.

In the production method, the ground ginger preparation may be performed by washing ginger and then grounding or squeezing the ginger in a phosphate buffer solution (PBS).

In the production method, the first to fourth centrifugation may be performed once or two or more times repetitively, and in particular, the third centrifugation may be performed once or two to five times repetitively, but is not limited thereto.

The production method may further include centrifugation of at least one phase.

According to an experimental example of the present disclosure, the ginger-derived extracellular vesicles were prepared by undergoing the first centrifugation performed once at 500 g for 10 minutes, the second centrifugation performed once at 2,000 g for 20 minutes using the supernatant obtained in the first centrifugation, the third centrifugation performed twice at 10,000 g for 30 minutes using the supernatant obtained in the second centrifugation, and the fourth centrifugation performed once at 100,000 g for 2 hours using the supernatant obtained in the third centrifugation.

It is possible to effectively obtain high-purity ginger-derived extracellular vesicles exhibiting excellent anticancer activity and uniform particle size via the production method as described above.

In addition, the production method may involve, in addition to the centrifugation method, the use of ultrafiltration, size exclusion chromatography, ion exchange chromatography, immunoaffinity capture, microfluidics-based isolation, exosome precipitation, total exosome isolation kit, or polymer based precipitation, but is not limited thereto. The production method may be carried out by further including isolation or purification methods well known in the art.

### Modes for Carrying Out Invention

Hereinafter, examples will be described in detail to help the understanding of the present disclosure. However, the following examples are merely illustrative of the content of the present disclosure, and the scope of the present disclosure is not limited to the following examples. The examples of the present disclosure are provided to more completely explain the present disclosure to those skilled in the art.

### EXPERIMENTAL EXAMPLE 1

### Characterization of Nanovesicles Isolated from Ginger

<Experiment Method>

1. Isolation of Nanovesicles from Ginger

Ginger used in an experimental example of the present disclosure was harvested from Andong, Gyeongsangbuk-do and purchased from Nonghyup. The purchased ginger was washed with clean water with no dirt left thereon. After washing, the skin of ginger was removed, and 150 g of ginger and 500 mL of 1× phosphate buffer saline (PBS) were put into a blender and ground for 5 minutes. The previously ground ginger was contained in 50 mL conical tubes in the volume of 50 mL each.

FIG. 1 schematically illustrates a method of isolating nanovesicles from ginger. Referring to this, the weight was constantly adjusted, centrifugation was carried out once using a centrifuge at 500 g for 10 minutes, and the supernatant was transferred to a new 50 mL conical tube to centrifuge the same once at 2,000 g for 20 minutes. Then, the supernatant was transferred to a new 50 mL conical tube to centrifuge the same twice at 10,000 g for 30 minutes each, and the supernatant was put in a new tube again for centrifugation at 100,000 g for two hours. The supernatant was discarded, and the remaining nanovesicle pellet were dried and dissolved in 1 mL of 1× PBS.

2. Nanoparticle Tracking Analysis (NTA)

Malvern's NanoSight was used to analyze extracellular vesicles (EVs) isolated from ginger. In the nanoparticle tracking analysis (NTA), the size and concentration of specific nanovesicles in the range of 50 nm to 1000 nm in diameter were measured in a liquid suspension.

3. Quantification of Ginger Nanovesicle Protein

Ginger nanovesicle protein was quantified by a bicinchoninic acid (BCA, Termo Fishers) method after pulverization in 1× RIPA solution.

4. Culture of Breast Cancer Cells and Lung Cancer Cells

MDA-MB-231 breast cancer cells and NCI-H460 lung cancer cells were obtained from an ATCC cell line. The cells were cultured in an incubator in the presence of 5% CO₂ at 37° C. by adding a DMEM medium, 10% fetal bovine serum, and 1% antibiotics for culture. Cells were replaced with media every 2-3 days.

5. Cell Proliferation Assay (MTT Assay)

MDA-MB-231 breast cancer cells and NCI-H460 lung cancer cells were respectively placed in a 96-well plate so that the number of cells per well becomes 2.5×10⁴ cells/mL and cultured in a DMEM growth medium for about 24 hours so that the cells are able to be attached to the plate. Then, the growth medium was removed and the ginger nanovesicles were treated under concentration conditions of 0, 10, 50, and 100 μg/mL (concentration conditions of 0, 1, 5, 10, 50, 100 μg/mL for lung cancer cells). Culture was carried out for about 24 hours in an incubator where 5% CO₂ is maintained at 37° C. Thereafter, a solution obtained by dissolving 2 mg/mL of MTT in 40 μL of PBS per well was added and left in an incubator at 37° C. for 2 hours. Next, the MTT solution was removed, and formazan formed by adding 100 μL of dimethyl sulfoxide (DMSO) per well was dissolved. The absorbance of completely dissolved formazan was measured at 595 nm with an ELISA-reader (Bio-Rad Lab-oratories Inc., Hercules, Calif., USA).

6. Cell Migration Assay

MDA-MB-231 breast cancer cells and NCI-H460 lung cancer cells were placed in a volume of 1 mL each in 6-wells and cultured in an incubator in the presence of 5% CO₂ at 37° C. After the cells were wounded, the healing of the cells was observed under a microscope.

7. Cell Invasion Assay

300 μL of MDA-MB-231 breast cancer cells suspended in a serum free medium was placed in an insert applied with a rehydrated polycarbonate membrane having a pore size of 8 μm, and 500 μL of medium containing 10% FBS was treated on an outer chamber, wherein a nutrient-rich environment that induces cell penetration was created on the outer chamber of the membrane. MDA-MB-231 breast cancer cells were treated with vascular endothelial growth factors (VEGFs) as a positive control at a concentration of 100 ng/mL, and ginger nanovesicles were treated for each concentration, followed by culture at 37° C. for 24 hours. After removing the unpenetrated cells, penetrated cells on the outer membrane were stained with a cell stain solution. The number of stained cells was counted.

8. Matrix Metallopeptidase 9 (MMP-9) ELISA Assay

The MMP-9 enzyme-linked immunosorbent assay (ELISA, R&D system) was conducted by labeling an antibody (or antigen) with an enzyme, and the enzymatic activity was identified using the company's protocol.

9. DID Labeling

MDA-MB-231 breast cancer cells were spread on an 8-well chamber slide at 1×10⁴ cells/well and cultured in a DMEM growth medium for about 24 hours. Thereafter, each well was treated with DID-labeled ginger nanovesicles, and the medium was removed after 24 hours, followed by washing with PBS and then 70% ethanol treatment. After 4′,6-diamidino-2-phenylindol (DAPI) staining, penetration of the ginger nanovesicles into the cells was identified with a fluorescence microscope.

10. Proteomics Assay

Ginger nanovesicles were treated with a trypsin buffer (500 ng/μL of trypsin, 50 mM of ammonium bicarbonate), followed by a reaction at 37° C. for 16 hours. After inactivation of trypsin by adding 5% formic acid, peptides were extracted using 25 mM of triethylammonium bicarbonate (TEABC) and acetonitrile (ACN). The extracted peptides were completely dried using a vacuum dryer, and right before the assay, the peptides were dissolved in an A buffer (0.1% formic acid) and analyzed by mass spectrometry. The data obtained by mass spectrometry was converted into a peak list (mgf file) using Mascot Distiller. Protein identification was conducted with the mgf file using the Mascot (Matrix Science; version 2.2.1) program.

11. Measurement of 2,2-Diphenyl-1-Picrylhydrazyl (DPPH)

DPPH was dissolved in ethanol, and vitamin C was dissolved in primary distilled water. Concentrations of vitamin C were adjusted to 1, 0.5, 0.25, 0.125, 0.0625, and 0.03125, and the vitamin C was placed in each 96-well along with ginger nanovesicles. After treating 150 μL of DPPH in each well, the absorbance was measured at 517 nm.

12. Statistical Processing

The results were obtained via three or more repeated experiments and represented as the mean±standard deviation for each sample concentration. In the significant difference test for each sample concentration group, the Student's t test was conducted in comparison with the control group, and a p<0.05 value was considered to be statistically significant.

<Experiment Results>

1. Identification of Ginger Nanovesicle Size

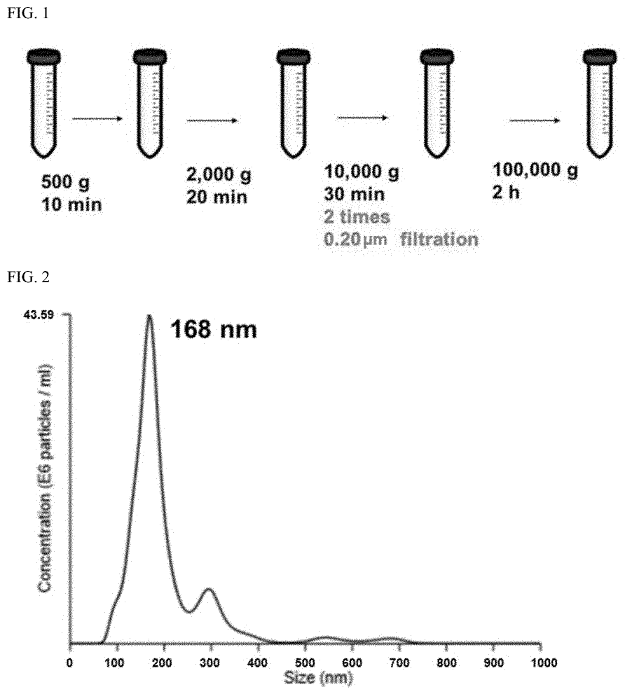

FIG. 2 is a graph of analyzing the size of ginger nanovesicle according to an experimental example of the present disclosure. Referring to this, as a result of nanoparticle tracking analysis (NTA) of the ginger nanovesicles isolated according to FIG. 1, the size of nanovesicles with the diameter of 168 nm was identified.

2. Confirmation of Inhibitory Effect of Ginger Nanovesicles on Breast Cancer Cell Survival

FIG. 3 is a graph showing the proliferation rate of breast cancer cells depending on the concentration of ginger nanovesicles according to an experimental example of the present disclosure.

In order to confirm the effect of ginger nanovesicles on the survival of MDA-MB-231 breast cancer cells, each concentration was treated, followed by observation via MTT assay. As shown in FIG. 3, when ginger nanovesicles were treated in a concentration of 0, 10, 50, and 100 μg respectively for 24 hours, the cell viability for each concentration was 100, 92, 88, and 79%, indicating a significant decrease in cell viability in a concentration-dependent manner from the concentration of 10 μg compared to the control group.

Therefore, it was confirmed that ginger nanovesicles induced concentration-dependent apoptosis in MDA-MB-231 breast cancer cells at the concentration of 10 μg or more, which was the same result as that ginger nanovesicles decreased the cell viability in a concentration-dependent manner in breast cancer cells. Through these results, it may be determined that the ginger nanovesicles exhibit the inhibitory effect on cancer cells.

3. Confirmation of Inhibitory Effect of Ginger Nanovesicles on Breast Cancer Cell Invasion

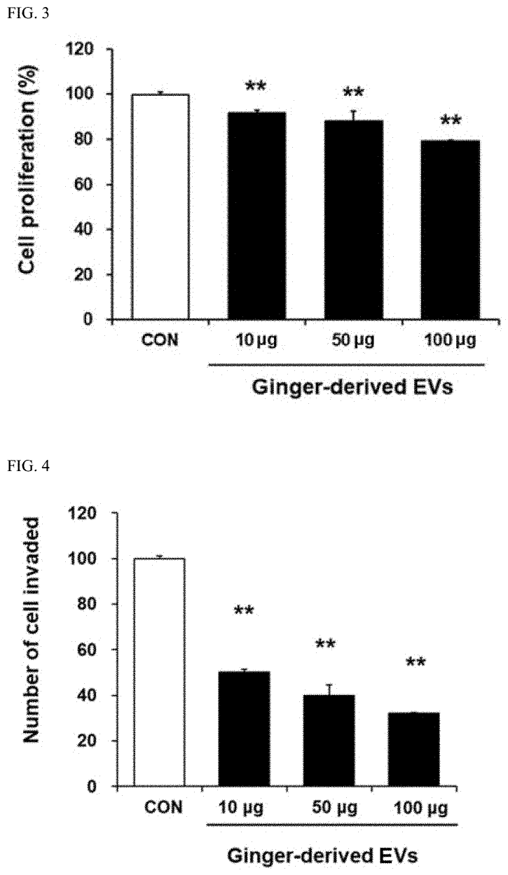

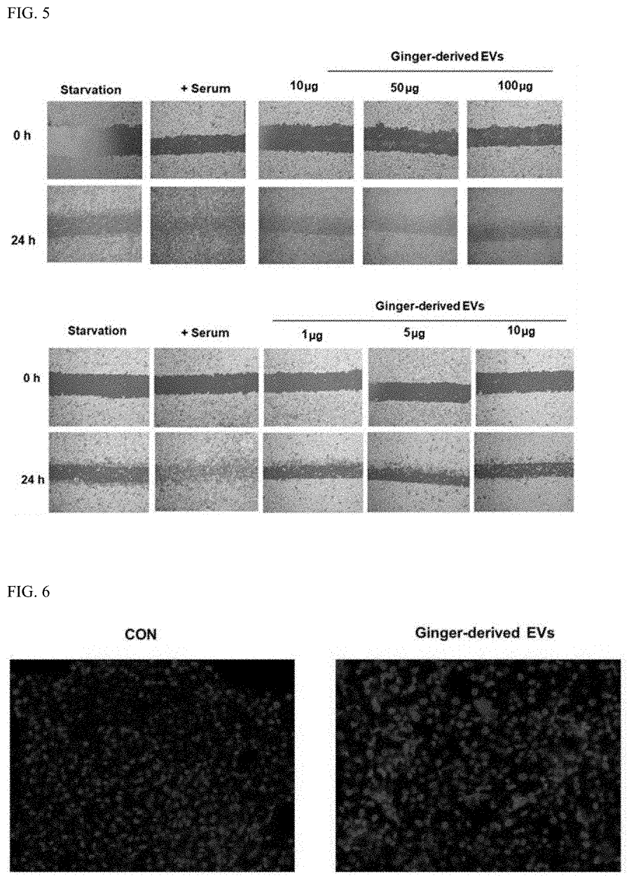

FIG. 4 is a graph showing the effect on the invasion of breast cancer cells depending on the concentration of ginger nanovesicles according to an experimental example of the present disclosure. Referring to this, it was shown that the invasion of MDA-MB-231 breast cancer cells was reduced in a concentration-dependent manner when the ginger nanovesicles were treated at a concentration of 0, 10, 50, and 100 μg respectively for 24 hours.

Therefore, it may be confirmed that the ginger nanovesicles inhibit cell invasion in a concentration-dependent manner in MDA-MB-231 breast cancer cells. Through these results, it may be determined that the ginger nanovesicles exhibit the inhibitory effect on cancer cells.

4. Confirmation of Inhibitory Effect of Ginger Nanovesicles on Breast Cancer Cell Migration

FIG. 5 shows a degree of migration of breast cancer cells depending on the concentration of ginger nanovesicles according to an experimental example of the present disclosure. Referring to this, it was shown that the migration of MDA-MB-231 breast cancer cells was reduced in a concentration-dependent manner when the ginger nanovesicles were treated at a concentration of 0, 1, 5, 10, 50, and 100 μg respectively for 24 hours.

Therefore, it may be confirmed that ginger nanovesicles inhibit cell migration of MDA-MB-231 breast cancer cells in a concentration-dependent manner. Through these results, it may be determined that ginger nanovesicles exhibit the inhibitory effect on cancer cells.

5. Confirmation of Effect of Ginger Nanovesicles on Breast Cancer Cell Internalization

FIG. 6 is images showing the penetration of ginger nanovesicles according to an experimental example of the present invention into breast cancer cells. Referring to this, when ginger nanovesicles were treated at a concentration of 10 μg for 24 hours, penetration into MDA-MB-231 breast cancer cells was identified.

Therefore, it may be determined that the ginger nanovesicles exhibit the inhibitory effect on cancer cells through the penetration of the ginger nanovesicles into MBA-MD-231 breast cancer cells.

6. Confirmation of the Inhibitory Effect of Ginger Nanovesicles on Lung Cancer Cell Survival

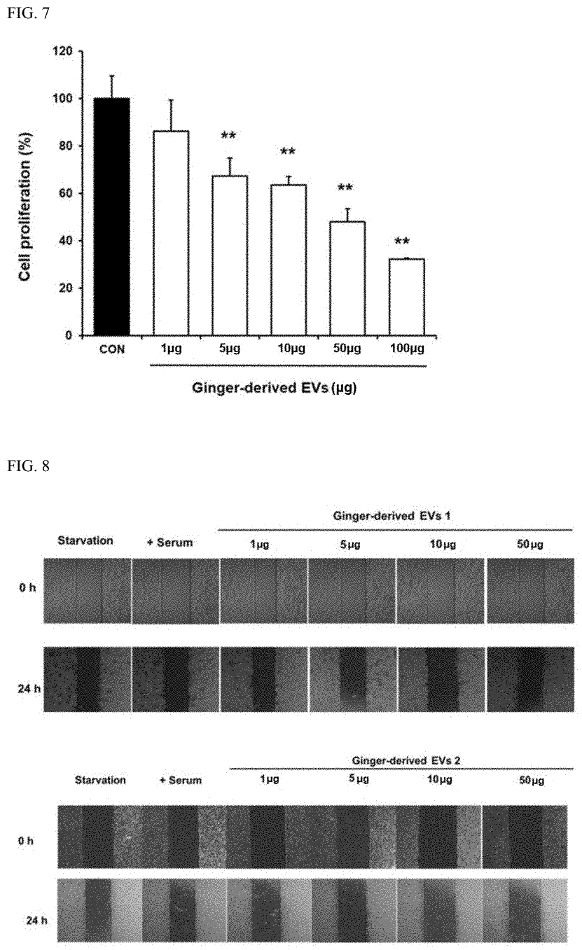

FIG. 7 is a graph showing the proliferation rate of lung cancer cells depending on the concentration of ginger nanovesicles according to an experimental example of the present disclosure.

In order to confirm the effect of ginger nanovesicles on the survival of NCI-H460 lung cancer cell, each concentration was treated, followed by observation via MTT assay. As shown in FIG. 7, when ginger nanovesicles were treated in a concentration of 0, 1, 5, 10, 50, and 100 μg respectively for 24 hours, the cell viability for each concentration showed a significant decrease in cell survival in a concentration-dependent manner from the concentration of 5 μg compared to the control group.

Therefore, it may be confirmed that the ginger nanovesicles induce concentration-dependent apoptosis in NCI-H460 lung cancer cells at a concentration of 5 μg or more, which was the same result as that the ginger nanovesicles reduced the cell viability in a concentration-dependent manner in lung cancer cells. Through the results, it may be determined that the ginger nanovesicles exhibit the inhibitory effect on cancer cells.

7. Confirmation of the Inhibitory Effect of Ginger Nanovesicles on Lung Cancer Cell Migration

FIG. 8 shows a degree of migration of lung cancer cells depending on the concentration of ginger nanovesicles according to an experimental example of the present disclosure. Referring to this, when the ginger nanovesicles were treated at a concentration of 0, 1, 5, 10, 50, and 100 μg respectively for 24 hours, the migration of NCI-H460 lung cancer cells was reduced in a concentration-dependent manner.

Therefore, it may be confirmed that the ginger nanovesicles inhibit cell migration in a concentration-dependent manner in NCI-H460 lung cancer cells, and through these results, it may be determined that the ginger nanovesicles exhibit the inhibitory effect on cancer cells.

8. Protein Identification of Ginger Nanovesicles

After preparing peptides through the trypsin digestion of ginger nanovesicles, proteomics assay was performed. As a result, five proteins were identified in the ginger nanovesicles as shown in Table 1 below. It was confirmed that enzyme proteins such as protease and cysteine proteinase as well as glyceraldehyde-3-phosphate dehydrogenase, which is an enzyme protein that plays an important role in energy production in photosynthesis of plants, were included in the ginger nanovesicles.

TABLE 1

| Protein Number | Protein Description | Protein score |
|---|---|---|
| 1 | Chain A, Protein (protease li) | 281 |
| 2 | RecName: Full = Zingipain-1; | 155 |
| | AltName: Full = Cysteine | |
| | proteinase GP-1 | |
| 3 | Cytosolic glyceraldehyde-3- | 84 |
| | phosphate dehydrogenase, partial | |
| | [Zingiber officinale] | |
| 4 | hypothetical chloroplast RF19 | 23 |
| | (chloroplast) [Zingiber officinale] | |
| 5 | teosinte branched1-like TCP | 21 |
| | transcription factor, partial | |
| | [Zingiber ottensii] | |

9. DPPH Radical Scavenging Ability for Each Concentration of Ginger Nanovesicles

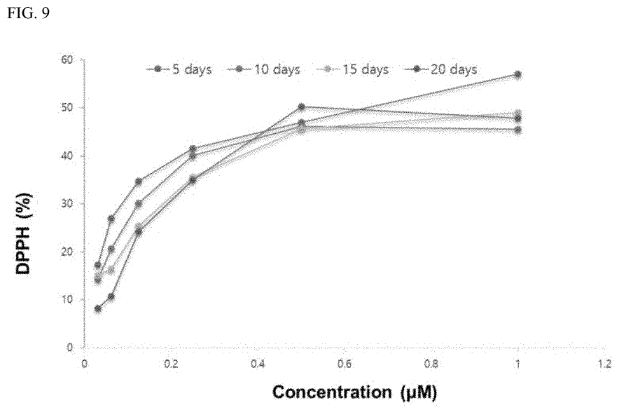

FIG. 9 is a graph identifying a DPPH radical scavenging ability of ginger nanovesicles according to an experimental example of the present disclosure. Referring to this, as a result of measuring DPPH radicals at intervals of 5 days, it was shown that the DPPH radical scavenging ability was decreased for each time.

Therefore, it may be confirmed that the ginger nanovesicles were oxidized over time, and the ginger nanovesicles had an antioxidant function. In addition, among methods of storing ginger, it was determined that storage after isolation into nanovesicles was important.

Although specific parts of the present invention have been described in detail above, it is clear for those skilled in the art that these specific descriptions are merely preferred example embodiments and the scope of the present invention is not limited thereto. In other words, the substantial scope of the present disclosure is defined by the appended claims and equivalents thereof.

1. An extracellular vesicle derived from ginger (Zingiber officinale), the extracellular vesicle having anticancer activity.

2. The extracellular vesicle of claim 1, wherein the extracellular vesicle is obtained by firstly centrifugating ground ginger at 250 to 750 g for 5 to 15 minutes, secondly centrifugating a supernatant obtained by the first centrifugation at 1,000 to 3,000 g for 10 to 30 minutes, thirdly centrifugating a supernatant obtained by the second centrifugation at 5,000 to 15,000 g for 15 to 45 minutes, and fourthly centrifugating a supernatant obtained by the third centrifugation at 50,000 to 150,000 g for 1 to 3 hours.

3. The extracellular vesicle of claim 1, wherein the extracellular vesicle is a nanovesicle having an average particle diameter of 50 to 300 nm.

4. The extracellular vesicle of claim 1, wherein the extracellular vesicle penetrates into a cancer cell.

5. The extracellular vesicle of claim 1, wherein the extracellular vesicle inhibits proliferation, migration, or invasion of cancer cells.

6. The extracellular vesicle of claim 1, wherein the extracellular vesicle comprises one enzyme protein or two or more enzyme proteins selected from the group consisting of protease, cysteine proteinase, and glyceraldehyde-3-phosphate dehydrogenase.

7. A pharmaceutical composition for preventing or treating cancer comprising the extracellular vesicle according to claim 1 as an active ingredient.

8. The pharmaceutical composition of claim 7, wherein the composition penetrates into cancer cells to inhibit proliferation, migration, or invasion of cancer cells.

9. The pharmaceutical composition of claim 7, wherein the cancer is a disease selected from the group consisting of breast cancer, lung cancer, liver cancer, stomach cancer, colorectal cancer, kidney cancer, bladder cancer, acute myeloid leukemia, acute lymphocytic leukemia, uterine cancer, ovarian cancer, laryngeal cancer, prostate cancer, thyroid cancer, head or neck cancer, brain cancer, and blood cancer.

10. A health functional food composition for preventing or ameliorating cancer comprising the extracellular vesicle according to claim 1 as an active ingredient.

11. A method of producing extracellular vesicles derived from ginger (Zingiber officinale), the method comprising:
preparing ground ginger;
firstly centrifuging the prepared ground ginger at 250 to 750 g for 5 to 15 minutes;
secondly centrifuging a supernatant obtained by the first centrifugation at 1,000 to 3,000 g for 10 to 30 minutes;
thirdly centrifuging a supernatant obtained by the second centrifugation at 5,000 to 15,000 g for 15 to 45 minutes; and
fourthly centrifuging a supernatant obtained by the third centrifugation at 50,000 to 150,000 g for 1 to 3 hours to obtain a pellet.

12. The method of claim 11, the centrifuging is performed once or two to five times repetitively.
